(19) United States
(12) Patent Application Publication
(10) Pub. No.: US 2024/0188571 A1
(43) Pub. Date: Jun. 13, 2024

(54) EMULSIFIABLE CONCENTRATE COMPOSITION AS WOOD PROTECTANT AND FOR TERMITE AND FUNGAL PATHOGENS CONTROL APPLICATIONS

(71) Applicant: Manuj NAYAR, Himachal Pradesh (IN)

(72) Inventor: Manuj NAYAR, Himachal Pradesh (IN)

(21) Appl. No.: 18/553,845

(22) PCT Filed: Apr. 4, 2022
(86) PCT No.: PCT/IN2022/050332

§ 371 Date: Oct. 4, 2023

(30) Foreign Application Priority Data
Apr. 10, 2021 (IN) 202111016875

(51) Int. Cl. A01N 65/06; A01N 25/04; A01P 3/00; A01P 7/04

(52) U.S. Cl. CPC A01N 65/06; A01N 25/04; A01P 3/00; A01P 7/04

(57) ABSTRACT

The present invention relates to Botanical based Cedar wood (Cedrus deodara) oil emulsifiable concentrate for protecting wood from termites and fungal pathogens and act as termiticide for pre and pre and post emergence termites. The composition comprises cedar wood oil as botanical active ingredient, single or a combination of emulsifiers for emulsification action and edible oil or non-edible oil or vegetable oil or mineral oil as solvent bulk medium. The composition of the present invention emulsifies to produce homogenous emulsion in water wherein the oil droplets tare emulsified. The compositions are suitable for spray applications or by brush application on wood surface and other target surfaces. The Emulsifiable concentrate is easier in dose preparation and effective in protecting wood from termites and decay fungi due to homogenous distribution of phytochemicals of cedar wood oil.

### RELATED PATENT APPLICATION

This application claims the priority to and benefit of Indian Patent Application No. 202111016875 filed on Apr. 10, 2021; the disclosure of which are incorporated herein by reference.

### FIELD OF THE INVENTION

The present invention relates to botanical based emulsifiable concentrate composition as wood protectant and for termite and fungal pathogens control applications. More particularly, the present invention relates to botanical based emulsifiable concentrate formulation compositions containing Cedar wood (Devadaru/Devdar—Cedrus deodara) oil as major active ingredient for protection of wood from termites and decay fungi with mineral oil or vegetable oil as diluents and an-ionic & non-ionic emulsifiers as dispersing agents, to produce stable emulsion upon dilution with water. The compositions may be applied on wood upon dilution with water, vegetable oil, mineral oil, etc. by brush or spray application. It acts as post and pre emergence termiticide and protects wood from termites and fungi.

### BACKGROUND OF THE INVENTION

Due to abundance in nature and versatility, wood is very important material used in construction of buildings, furniture and many items for day to day use like handles of construction tools, decoration items etc. It has important role in human life and global economy. Wood is a natural composite of cellulose fibres that are embedded in matrix of lignin. Because of organic nature, wood is sensitive to attack from insects like termite and microbes like fungi. Termite attack is the major factor for decay of wood used in buildings, furniture and other articles of human usage. The termite attack cause huge monetary losses globally.

Termites, the foremost enemy of wooden structures in buildings although tiny, remain a formidable threat to the modern construction projects. Hardwood and Plywood used extensively for the interiors are highly susceptible for termite attack, making millions of rupees invested go waste in quite a short span of time.

Pre-construction anti-termite insecticidal management although extremely popular, fail to provide a permanent solution. The effectiveness of the insecticides remains viable for shorter period of time. Once, the repellent effect is gone, the ever-persuasive termites swarm into the wooden structures unknowingly causing greater damage from inside. Ultimately, when the damage gets visible, it is irreparable and causes loss of entire structure.

The management become more difficult as synthetic chemical insecticides and wood preservatives cannot be used on wooden structures because of their harmful nature. Many of these chemical preservatives and insecticides are nerve poisons leave toxic residues and obnoxious odour. Further, as wood and plywood are used extensively for interiors as like wardrobes etc., which are in daily human contact, their insecticidal treatment becomes unsafe.

Plants extracts with pesticide properties have been considered and investigated for decades as alternatives to synthetic pesticides. The reason of consideration of plants as pesticides is because they are more environmentally safer and have biodegradability. Plant derived extracts; essential oils and fixed oils contain a multiple bio-actives including alkaloids, phenolics, terpenes etc., and can be utilized in management of various pests, termites and fungi. A large number of botanical-based products remains restricted and little progress has been made to develop new products. The degradation and volatilization resulting in loss of bioactive compounds are the main drawback that reduces the efficiency of plant-based products.

Consequently, their potential is not well accepted. Development of suitable formulations may provide better stability, application and efficacy against target insects. The utilization of such plant extracts, essential oils and development of their formulations for controlling termites and microbes may be beneficial and safer alternative to chemical products. The development of suitable formulation from crude extract would be much easier for spray applications and will provide better shelf life and bio-efficacy.

The chemicals like Penta Chloro phenol, Acid Copper Chromate, N-Cyclo hexa decyldiaeniumdioxlyl copper, Chromated Copper Arsenate, Copper azole, Creosote, Propiconazole, Triadimefon etc. have been used as wood preservatives to protect wood from termites and fungi. The usage of chemical based termiticides and wood preservatives pose risks to the safety of user, termites and microbes establish resistance to almost every synthetic chemical upon prolonged use. There remains a need in the art for a botanical active ingredient based Emulsifiable Concentrate formulation that readily disperses in water and, maintains physical and chemical stability during long-term storage under environmentally stressful conditions, and exhibits excellent protection of wood from termites.

Cedarwood oil and cedrol have been described for efficacy against ants, ticks in Journal (Bioactivity of Cedarwood oil and cedrol against Arthropod pests: F. J. Eller et al, 2014 Environ. Entomol: 43(3) 762-766). However, this prior art is limited to the use of these Carbon dioxide derived Cedarwood oil and cedrol providing repellence and efficacy against arthropod, pests, ants, ticks etc.

Further F. J. Miller et. al in Journal (International Bio-deterioration and Biodegradation 2018, Vol. 129: 95-101) have described utilization of hexa decyl ammonium chloride amylase inclusion complex preparation with cedar wood oil and treatment of wood against termite and fungi. This prior art is limited to the preparation of hexa decyl ammonium chloride amylase inclusion complex prepared by complex process at high pressure and high temperature up to 140° C. and use of harsh chemicals and acid for making the complex with cedarwood oil. This prior art is limited to studies on the prepared complex as termiticidal and fungicidal properties and does not provide a ready to use environment friendly product.

All the prior arts are limited to the cedar wood oil in pure form or preparation of its complexes by chemical processes and do not provide a ready to use product for wood preservation and protection applications. The cedar wood oil in pure form is a viscous oil, which needs to be diluted with water so that small amount of active ingredient may be homogeneously distributed over large surface area of wood to get the effective protection from termites and decay fungi. The oil in pure form is viscous and concentrated, thus it cannot be applied by convenient application methods at the target sites. The present invention describes Emulsifiable Concentrate formulation of cedar wood oil (Cedrus deodara), which upon dilution with water provides an emulsion. This emulsion can be conveniently applied by spray or brush on wood surface to protect wood from termite and fungi.

The preparation of formulations is essential for effective implementation of active ingredient for producing efficacy at site. The cedar wood oil may not be applied in pure form due to high cost, viscosity and higher concentration and undilutability. Thus, there is need for, a formulation technique, which can maintain the shelf life of phytochemical constituents of botanical active ingredient and also provide desired efficacy. The emulsifiable concentrate formulation described in the present invention is prepared by simple mixing and a ready-to-use product, which upon diluting with water gives stable emulsion for applying by spray or brush for protecting wood from termites and decay fungi.

### OBJECTS OF THE INVENTION

The primary object of the invention is to produce an Emulsifiable Concentrate formulation of Cedarwood (Devadaru/Devdar—Cedrus deodara) oil suitable for brush or spray or application after dilution in water upon wood for protecting wood from termites and decay fungi.

Another object of the invention is to provide a stable composition prepared using optimized ratios of cedar wood oil single or combination, emulsifiers and edible oils or non-edible oils or vegetable oils as solvents in the formulation.

Another object of the invention is to provide stable Emulsifiable Concentrate composition, which is stable on long-term storage.

Yet another object of invention is to provide composition that emulsifies in water producing homogenous emulsion for uniform distribution on target area.

One more object of the invention is to provide easy application of cedar wood oil emulsion with homogenous distribution of bioactive constituents, to have adequate protection and bio-efficacy against target termites and wood decay fungi.

### SUMMARY OF THE INVENTION

The present invention provides an Emulsifiable Concentrate formulation containing Cedarwood (Cedrus deodara) oil, a solvent selected from edible oil or non-edible oil or vegetable oil, or their combination and an emulsifier selected from non-ionic and an-ionic emulsifier, or a combination thereof. The formulation of the present invention is prepared by low shear mixing of cedar wood oil, solvent and emulsifier. The resultant composition readily disperses in water to provide a stable emulsion suitable for application on wood for control and prevention of termite and wood decay fungi.

The compositions are effective in control and prevention of termite and fungi infestation on wood. The composition offers various advantages like ease in handling, volumetric measurement and dose preparation: and good bio-efficacy. As the compositions are based on botanical based active ingredients, these are safe towards non-target organisms and do not pose risk of environmental contamination, dermal toxicity to the human and non-target organisms. The compositions may also be safely used in agriculture and organic farming for termite control. The bio-efficacy testing results indicate that these stable oil-based dispersions of the present invention, has very good bio efficacy and significantly reduced termites and protected wood from infestation of fungi. The compositions are also storage stable, and provide stable emulsion in water for application on wood surface and very effective in protecting wood from termites and wood decay fungi.

Accordingly, one aspect of the present invention is to provide an Emulsifiable Concentrate composition of Cedar wood (Cedrus deodara) oil comprising:
(i) Cedar wood oil: as wood preservative, protectant, termiticide and fungicide;
(ii) at least one emulsifier selected from ionic or non-ionic emulsifiers or a combination thereof; and
(iii) solvent bulk medium selected from edible oil, non-edible oil, vegetable oil, mineral oils or a combination thereof.

The said Cedar wood oil extracted from wood of cedar (Devdar/devadaru), is in an amount of 5-50% w/w.

The said emulsifier is selected from ethoxylated castor oil, tristeryl phenol ethoxylate, fatty alcohol ethoxylate, calcium alkyl benzene sulphonate, triestryl phenol phosphate ester, nonyl phenol ethoxylate, or combination thereof, and is in an amount of 3-25% w/w.

The said solvent or bulk medium oil phase is carrier oil selected from soya bean oil, sunflower oil, rice bran oil, ground nut oil, castor oil, mineral oils and C9 Aromatic Hydrocarbons Solvents or mixture thereof, and is in an amount of 20-90% w/w.

The said wood protectant, termiticide, fungicide Cedar wood oil, as an active ingredient, protects wood from termites, fungi and also acts as termiticide for wood and termite management applications.

The said Emulsifiable concentrate is stable at lower and elevated temperature ranges of 0° C. to 54° C., for a storage period of 12 months.

Another aspect of the present invention is to provide an Emulsifiable Concentrate composition of Cedar wood (Cedrus deodara) oil prepared by a process comprising the steps of:
i) premixing Cedar wood oil with solvent medium selected from edible oil, non-edible oil, vegetable oil, mineral oil or a combination thereof, at low shear mixing at 40-50 rpm;
ii) adding at least one emulsifier selected from ionic or non-ionic emulsifiers or a combination thereof to the solution of step (i), with continued low high shear mixing at a speed of 40-50 rpm, to prepare emulsifiable concentrate composition; and
iii) optionally filtering the emulsified concentrate composition of step (ii) for further purification.

The said solvent or bulk medium oil phase in step (i) is carrier oil selected from soya bean oil, sunflower oil, rice bran oil, ground nut oil, castor oil, mineral oils and C9 Aromatic Hydrocarbons Solvents or mixture thereof.

The said emulsifier in step (ii) is selected from ethoxylated castor oil, tristeryl phenol ethoxylate, fatty alcohol ethoxylate, calcium alkyl benzene sulphonate, triestryl phenol phosphate ester, nonyl phenol ethoxylate, or combination thereof.

### DETAILED DESCRIPTION OF THE INVENTION

Detailed embodiments of the present invention are disclosed herein below. However, it is to be understood that the disclosed embodiments are merely exemplary of the invention, which can be embodied in various forms. The scope of the invention is not limited to the disclosed embodiments and terms and phrases used herein are not intended to be limiting but rather to provide an understandable description of the invention.

The present invention provides a novel Emulsifiable Concentrate formulation of cedar wood oil. The edible or non-edible or vegetable oils acts as solvent/bulk medium and the selected emulsifier combination produces stable composition of cedar wood oil formulation. The composition is homogenized using low shear mixer and is stable on long-term storage. The composition prepared from the description is effective in controlling and preventing wood from termites and fungi. The harsh chemicals being used as wood preservatives are not safe to user and environment. Thus, there is need for new botanical based formulation, which are safe to humans, non-target organisms and environment and which can maintain good shelf life of phytochemical constituents of botanical based products and also provide adequate efficacy.

In an exemplary embodiment of the present invention, the stable cedar wood oil Emulsifiable Concentrate formulation is prepared from composition comprising of:
i) Cedar wood (Devdar/Devadaru—Cedrus deodara) oil as a botanical termiticide, fungicide and wood protectant,
ii) at least one emulsifier selected from ionic or non-ionic emulsifiers or a combination thereof; and
iii) solvent bulk medium selected from edible oil, non-edible oil, vegetable oil, mineral oils or a combination thereof.

In another exemplary embodiment, the composition comprises emulsifying agents in an amount of 3-25% w/w, cedar wood oil in range of 5-50% w/w, and Vegetable oils or solvents in the range of 20-90% w/w.

In an exemplary embodiment of the invention, the emulsifiers are selected form ethoxylated castor oil, tristeryl phenol ethoxylate, fatty alcohol ethoxylate, calcium alkyl benzene sulphonate, tristeryl phenol phosphate ester or combinations thereof. These emulsifiers perform dual function of stabilizing emulsifiable concentrate in concentrated form and to provide instant emulsification in water at the time of spray or brush application. Further, the continuous phase, vegetable oil is selected from soya bean oil, sunflower oil, rice bran oil, castor oil, or mixtures thereof or mineral oils like C-IX or aromax.

In one embodiment, the composition of the present invention is stable at lower and elevated temperature ranges i.e. 0° C. to 54° C. The performance of given composition is not affected during storage stability.

In another embodiment, the composition of the present invention, greatly improves physical and chemical stability of bioactive constituents present in cedar wood oil. The formulation of invention provides effective control and prevention from termites and fungi, which are responsible for significant losses to wood used in furniture, buildings and various items. The composition is effective in controlling pre and post emergence of termites and prevention from wood decay fungi. The main advantage of this formulation is that it provides easy application of the botanical based active i.e., cedar wood oil with homogenous distribution of bioactive constituents, which is desired for adequate bio-efficacy against target termites and at lower does without causing any harm to user i.e., human beings and non-target organisms like pets, birds etc. The present compositions can effectively protect wood from termite attack and infestation of decay fungi.

Further, the stable emulsifiable concentrate compositions of the present invention can be prepared by ingredients that are cost effective, easily available and by using simple and industrially feasible process of low shear mixing at 40-50 rpm to get homogeneously mixed product.

In an exemplary embodiment, the stable, emulsifiable concentrate formulation of cedar wood oil is prepared by a process comprising the steps of:
i) premixing of Cedar wood oil with edible or non-edible oil being taken as bulk medium in the composition by using a low shear mixer at 40-50 rpm,
ii) addition of emulsifiers with continued low high shear mixing at a speed of 40-50 rpm to ensure homogeneous mixing of ingredients, and
iii) filtration to remove dirt particle, if any to get final product.

The Emulsifiable concentrate is an oil-based formulation containing the active and inert ingredients which is applied as an emulsion after dilution in water. An oil like mineral oil or preferably vegetable oil, is used to solubilize the active ingredient i.e., cedar wood oil. After dilution in water, the formulation emulsifies in water producing homogenous suspension suitable for application by convenient methods like spraying or by brushing. Furthermore, vegetable oil functions as adjuvant to provide enhanced bio-efficacy when it is diluted with water for applying on wood. By choosing the appropriate oil base and surfactants, the active phytochemicals can remain physically and chemically stable for an extended period of time, ensuring a long shelf life for the product.

The present invention is explained in detail with reference to the following examples described below, which are given for the purpose of illustration only and are not intended to limit the scope of the invention.

### Examples

The Cedar wood oil is obtained from commercial sources and local Vendors in India.

### 1. General Composition of Emulsifiable Concentrate:

The Emulsifiable concentrate are prepared according to following concentration range of the ingredients given in table-1 and role of each ingredient used is mentioned in table 2

TABLE 1
General composition of Emulsifiable concentrate

| Ingredients | % w/w |
|---|---|
| Emulsifiers | 3-20 |
| Cedar wood oil | 5-40 |
| Edible or non-edible Oil or vegetable oil or mineral oil | Q.C |
| Total | 100 |

TABLE 2
Role of ingredients

| Ingredients | Role |
|---|---|
| Emulsifiers | Emulsification of formulation upon |
| | dilution in water |
| Cedar wood oil | Active ingredient |
| Edible oil or non-edible Oil or | Solvent/Bulk medium |
| vegetable oil or mineral oil | |

### 2. Preparation of Cedar Wood Oil Emulsifiable Concentrate Formulation

The cedar wood oil is added into optimized Vegetable or Edible or non-edible or mineral oil or their combinations Oil under low shear mixing at 40-50 rpm to get clear homogeneous solution. The optimized emulsifiers are added into the solution and low shear mixing is continued followed by filtration to remove dirt particle, if any, to get homogeneous emulsifiable concentrate product. Different compositions were prepared, some are described below in examples 1-8.

### Example-1

TABLE 3
Ingredients and concentration ranges of Composition 1

| Ingredients | % w/w |
|---|---|
| Ethoxylated castor oil | 5 |
| Calcium alkyl benzene sulfonate | 5 |
| Cedar wood oil | 20 |
| Sunflower Oil | 70 |
| Total | 100 |

### Example-2

TABLE 4
Ingredients and concentration ranges of Composition 2

| Ingredients | % w/w |
|---|---|
| Tristeryl phenol ethoxylate | 7.5 |
| Fatty alcohol ethoxylate | 2.5 |
| Cedar wood oil | 10 |
| Soya been Oil | 80 |
| Total | 100 |

### Example-3

TABLE 5
Ingredients and concentration ranges of Composition 3

| Ingredients | % w/w |
|---|---|
| Fatty alcohol ethoxylate | 8 |
| Calcium alkyl benzene sulfonate | 7 |
| Cedar wood oil | 25 |
| Soya bean Oil | 30 |
| Sunflower oil | 30 |
| Total | 100 |

### Example-4

TABLE 6
Ingredients and concentration ranges of Composition 4

| Ingredients | % w/w |
|---|---|
| Triestryl phenol phosphate ester | 8 |
| Ethoxylated Nonyl phenol | 9 |
| Cedar wood oil | 30 |
| Sunflower Oil | 14 |
| Rice bran oil | 39 |
| Total | 100 |

### Example-5

TABLE 7
Ingredients and concentration ranges of Composition 5

| Ingredients | % w/w |
|---|---|
| Ethoxylated Castor oil | 16 |
| Cedar wood oil | 35 |
| Soya been oil | 49 |
| Total | 100 |

### Example-6

TABLE 8
Ingredients and concentration ranges of Composition 6

| Ingredients | % w/w |
|---|---|
| Triestryl phenol phosphate ester | 9 |
| Ethoxylated Nonyl phenol | 10 |
| Cedar wood oil | 36 |
| Castor oil | 45 |
| Total | 100 |

### Example-7

TABLE 9
Ingredients and concentration ranges of Composition 7

| Ingredients | % w/w |
|---|---|
| Calcium alkyl benzene sulfonate | 8 |
| Ethoxylated Nonyl phenol | 10 |
| Cedar wood oil | 30 |
| Groundnut oil | 52 |
| Total | 100 |

### Example-8

TABLE 10
Ingredients and concentration ranges of Composition 8

| Ingredients | % w/w |
|---|---|
| Nonyl phenol ethoxylate | 12 |
| Cedar wood oil | 25 |
| Mineral oil (Solvent CIX) | 40 |
| Mineral oil (Aromax) | 23 |
| Total | 100 |

### 3. Physico-Chemical Parameters:

The pH, persistent foam, emulsion stability of composition was studied

### Methodology:

i) pH: pH of 1% emulsion in distilled water was measured with digital pH meter.
ii) Persistent foam: Persistent foam was determined by—2 ml of formulation was diluted with standard hard water (500 ppm) to make 100 ml in measuring cylinder. Cylinder was given 30 inversions Top of cylinder was observed for presence of foam, if any.
iii) Emulsion stability: 2 ml formulation was diluted in 100 ml standard hard water (500 ppm) in measuring cylinder and observed for any sedimentation, creaming or phase separation.
iv) Storage stability: The formulation was stored at 54° C., for 14 days and observed for physical changes.

### 4. Bio Efficacy Studies Against Termites:

### Methodology:

The formulation was evaluated under laboratory conditions following standard protocols for its contact and surface toxicity, against termite, Odontotermes sp. to simulate real field conditions. In the topical (direct contact toxicity) assay, the bio-termiticide was sprayed directly on the termites and mortality (death), repellence (moving away) or anti-feedant (non-feeding) effects were studied. This assay was carried out in simulation of curative management viz., post-infestation management. In the surface toxicity assay the surface on which the termites walk over was sprayed, termites were allowed to move on the treated surface later and mortality (death), repellence (moving a way) or anti-feedant (non-feeding) effects were studied. This assay was carried out in simulation of preventive management viz., pre-infestation management. These two methods were planned as they suffice the current needs of pre- and post-infestation termite management.

Both the topical and surface toxicity bioassays were carried out using planar arena assay method (Chouvenc et al. 20112011, Planar Arenas for Use in Laboratory Bioassay Studies of Subterranean Termites (Rhinotermitidae). Florida Entomologist, 94(4): 817-826) with minor modifications. Seven concentrations of the cedar wood oil Emulsifiable concentrate formulation, standard check Chlorpyriphos and untreated control were used in the bioassay.

V₁N₁=V₂N₂
Where,
   V₁=Volume of the Stock needed to be taken
   N₁=Concentration of the Stock (100%)
   V₂=Volume of the treatment to be prepared
   N₂=Concentration of the treatment needed

Market sample of Chlorpyriphos was used as standard check for comparing the effect of this cedar wood oil botanical based emulsifiable concentrate formulation.

The experiment was initiated by collecting termites from already infested woods @ 50 numbers per glass jar (100 ml capacity) painted black. The lid was pierced with a pinhole to provide aeration. These jars were provided with pre-moistened, partially decayed wooden chips and wet soil as food to termites. These jars were kept inside environmental chambers at a temperature of 26±1° C. and a relative humidity of 80% under complete darkness.

The treatment planar arena was prepared from commercial hardwood plywood (8 mm thickness). One square foot ply wood pieces were cut out and soaked in water over night. Then they were air-dried for 48 hours under room temperature. Plastic spacers were placed on top of the pre-soaked plywood so as to leave a depth of 0.75 cm, if covered from the top. Mylar sheets were cut out in such a way that they fit the top of these pre-soaked plywood pieces. The mylar sheet was pierced with small pinholes for aeration and a punch hole was made using single paper punch machine in the centre for release of termites.

After 72 hours, 25 worker termites were collected from the glass jar and released into treatment planar area along with soil and pre-digested wood debris present in the jar. For topical assay, the entire setup along with termites was sprayed with 200 ml of each concentration of bio-termiticide using an atomizer. The top of the plywood was closed immediately with mylar sheet cover and the setup was kept inside environmental chamber at a temperature of 26±1° C. and a relative humidity of 80% under complete darkness. Similarly, chlorpyriphos treatment and untreated control were maintained. Five such replications were maintained per treatment. After 24 hours, death and movement away from the planar area were observed and percent mortality and repellence worked out.

For surface toxicity assay, the planar area was first sprayed with the respective concentrations of cedar wood oil emulsifiable concentrate formulation, standard check separately and after one hour, 25 worker termites along with food were released. Similarly, percent mortality and repellence worked out.

5. Bio Efficacy Evaluation on Protection of Wood from Decay Fungi

Blocks of Marandi wood of size 30×15×4 cm (L×B×W) were taken for the experiment. The emulsion of cedar wood oil emulsifiable concentrate formulation in water was sprayed on two replicates of wooden blocks using a band sprayer. No emulsion was applied on untreated control. Market sample of Propiconazole was used as standard check for comparing the effect of this botanical cedar wood oil emulsifiable concentrate formulation. The blocks after application and untreated control were kept in open space for a period of one year starting from the month of November up to the month of October to subject the blocks to winter, summer and rainy season. Observations on the wooden blocks were taken to check the infestation of fungi and damage caused by fungi.

### 6. Results:

### Results of Physico-Chemical Parameters Analysis:

TABLE 11
Physico-chemical Quality parameters (Initially)

| | | Persistent | |
|---|---|---|---|
| Compositions | pH | Foam | Emulsion stability |
| Composition 1 | 7.2 | 0 ml | No sedimentation, creaming, phase |
| | | | separation |
| Composition 2 | 7.1 | 0 ml | No sedimentation, creaming, phase |
| | | | separation |
| Composition 3 | 6.9 | 0 ml | No sedimentation, creaming, phase |
| | | | separation |
| Composition 4 | 7.3 | 0 ml | No sedimentation, creaming, phase |
| | | | separation |
| Composition 5 | 6.9 | 0 ml | No sedimentation, creaming, phase |
| | | | separation |
| Composition 6 | 7.0 | 0 ml | No sedimentation, creaming, phase |
| | | | separation |
| Composition 7 | 7.2 | 0 ml | No sedimentation, creaming, phase |
| | | | separation |
| Composition 8 | 7.0 | 0 ml | No sedimentation, creaming, phase |
| | | | separation |

TABLE 12
Physico-chemical Quality parameters
(After one year of storage at ambient temperature)

| | | Persistent | |
|---|---|---|---|
| Compositions | pH | Foam | Emulsion stability |
| Composition 1 | 7.2 | 0 ml | No sedimentation, creaming, phase |
| | | | separation |
| Composition 2 | 7.0 | 0 ml | No sedimentation, creaming, phase |
| | | | separation |
| Composition 3 | 6.8 | 0 ml | No sedimentation, creaming, phase |
| | | | separation |
| Composition 4 | 7.4 | 0 ml | No sedimentation, creaming, phase |
| | | | separation |
| Composition 5 | 6.8 | 0 ml | No sedimentation, creaming, phase |
| | | | separation |
| Composition 6 | 7.2 | 0 ml | No sedimentation, creaming, phase |
| | | | separation |
| Composition 7 | 7.3 | 0 ml | No sedimentation, creaming, phase |
| | | | separation |
| Composition 8 | 7.1 | 0 ml | No sedimentation, creaming, phase |
| | | | separation |

The pH of compositions as shown in table 11 and 12 were found to be near to neutral. There was no major change in pH, persistent foam and emulsion stability after one year of storage at ambient temperatures. The results indicate that composition is stable for minimum one year storage.

TABLE 13
Storage stability of formulation

| Time | Phase separation | Settling |
|---|---|---|
| 0 | Uniform with no separation | No Settling |
| After 14 days (54° C.) | Uniform with no separation | No Settling |

The formulation was stable at Accelerated Temperature storage studies at 54° C. for 14 days (equivalent to two years at ambient temperature). There was no phase separation, settling occurred after storing for 14 days. These results indicate that the composition is stable for long-term storage of two years.

### Results of Bio-Efficacy Against Termites:

The results of bio-efficacy against termites are given in Tables 14 & 15. The results of both the bioassays clearly indicated the effectiveness of the formulation in the management of termites. When percent mortality was considered, the product performed even better than the standard check, chlorpyriphos at concentrations tested (from 30 ml/lt. concentration in topical assay and surface toxicity assay). The product at 25 ml/lt. concentration performed on par with the insecticide chlorpyriphos, indicating its effectiveness. This underlines the practical use of the product in pre and post-infestation management of termites. At concentrations below 15 ml/lt., the mortality was less. However, the percent repellence was more than chlorpyriphos in all concentrations. this indicated multi-pronged anti-termite action of the formulation described in the invention. This gains importance due to the fact that this prevents the resistance development in termites, a problem encountered w % bile using insecticides repeatedly.

Further, when the effect of this formulation compared among methods of bioassay, mortality effect in surface toxicity assay was found inferior than topical assay. However, the repellence action was surprisingly more even to the tune of 24 percent. Hence, the combined anti-termite efficacy is greater than the standard check, chlorpyriphos. Such effect highlights its advantageous use the product in pre-infestation management of termites and therefore may be effectively used for wood protection.

Further, this formulation has a dose dependent effect providing increased mortality with increased dose. This substantiates the utility of the product in restoration of wooden structures damaged due to heavy termite infestation.

TABLE 14
Effect of Cedar wood oil emulsifiable concentrate
formulation against termites under topical assay

| Treatment | | Percent | Percent |
|---|---|---|---|
| No. | Treatment description | Mortality* | repellence* |
| 1 | Cedarwood oil Emulsifiable | 48 (43.85)^{e} | 12 (20.27)^{c} |
| | Concentrate @10 ml/lit. | | |
| 2 | Cedarwood oil Emulsifiable | 60 (50.77)^{d} | 16 (23.58)^{b} |
| | Concentrate @15 ml/lit. | | |
| 3 | Cedarwood oil Emulsifiable | 72 (58.05)^{c} | 24 (29.33)^{a} |
| | Concentrate @20 ml/lit. | | |
| 4 | Cedarwood oil Emulsifiable | 92 (73.57)^{b} | 4 (11.54)^{d} |
| | Concentrate @25 ml/lit. | | |
| 5 | Cedarwood oil Emulsifiable | 96 (78.46)^{a} | 4 (11.54)^{d} |
| | Concentrate @30 ml/lit. | | |
| 6 | Cedarwood oil Emulsifiable | 96 (78.46)^{a} | 4 (11.54)^{d} |
| | Concentrate @35 ml/lit. | | |
| 7 | Cedarwood oil Emulsifiable | 96 (78.46)^{a} | 4 (11.54)^{d} |
| | Concentrate @40 ml/lit. | | |
| 8 | Chlorpyriphos @ 25 ml/lit. | 92 (73.57)^{b} | 0 (0.29)^{e} |
| 9 | Untreated control | 0 (0.29)^{f} | 0 (0.29)^{e} |

| | | |
|---|---|---|
| S.E(d) | 1.34 | 0.26 |
| C.D (=0.05) | 2.82 | 0.54 |

(*Mean of five replications, Values in parentheses are arc sine transformed values Values with different alphabets within a column differ significantly)

TABLE 15
Effect of Cedar wood oil emulsifiable concentrate formulation under
surface toxicity assay against termites under topical assay

| Treatment | | Percent | Percent |
|---|---|---|---|
| No. | Treatment description | Mortality* | repellence* |
| 1 | Cedarwood oil Emulsifiable | 40 (39.23)^{e} | 20 (26.57)^{b} |
| | Concentrate @10 ml/lit. | | |
| 2 | Cedarwood oil Emulsifiable | 56 (48.45)^{d} | 20 (26.57)^{b} |
| | Concentrate @15 ml/lit. | | |
| 3 | Cedarwood oil Emulsifiable | 68 (55.55)^{c} | 24 (29.33)^{a} |
| | Concentrate @20 ml/lit. | | |
| 4 | Cedarwood oil Emulsifiable | 80 (63.43)^{b} | 12 (20.27)^{c} |
| | Concentrate @25 ml/lit. | | |
| 5 | Cedarwood oil Emulsifiable | 80 (63.43)^{b} | 12 (20.27)^{c} |
| | Concentrate @30 ml/lit. | | |
| 6 | Cedarwood oil Emulsifiable | 84 (66.42)^{a} | 8 (16.43)^{d} |
| | Concentrate @35 ml/lit. | | |
| 7 | Cedarwood oil Emulsifiable | 84 (66.42)^{a} | 8 (16.43)^{d} |
| | Concentrate @40 ml/lit. | | |
| 8 | Chlorpyriphos @ 25 ml/lit. | 80 (63.43)^{b} | 4 (11.54)^{e} |
| 9 | Untreated control | 0 (0.29)^{f} | 0 (0.29)^{f} |

| | | |
|---|---|---|
| S.E (d) | 1.12 | 0.39 |
| C.D (=0.05) | 2.35 | 0.82 |

(*Mean of five replications, Values in parentheses are arc sine transformed values, Values with different alphabets within a column differ significantly)

The results indicate that cedar wood oil Emulsifiable concentrate formulation described in this invention is suitable for the pre- and post-infestation effective management of termites and it will protect wood from attack of termites.

Results of Bio Efficacy Evaluation on Protection of Wood from Decay Fungi

No infestation of the fungi on the blocks treated by cedar wood oil emulsifiable concentrate formulation and Propiconazole was observed. However, on the untreated control, the infestation of Fusarium oxysporum fungus on wood surface was observed. The results indicate that the cedar wood oil emulsifiable concentrate formulation described in the invention is effective in protecting wood from attack of decay fungi and performed equivalent to synthetic chemical Propiconazole.

I claim:

1. An Emulsifiable concentrate composition of Cedar wood (Cedrus deodara) oil comprising:
(i) Cedar wood oil;
(ii) at least one emulsifier selected from ionic or non-ionic emulsifiers or a combination thereof; and
(iii) solvent bulk medium selected from edible oil, non-edible oil, vegetable oil, mineral oils or a combination thereof.

2. The composition as claimed in claim 1, wherein said Cedar wood oil is in an amount of 5-50% w/w.

3. The composition as claimed in claim 1, wherein said emulsifiers are selected from ethoxylated castor oil, tristeryl phenol ethoxylate, fatty alcohol ethoxylate, calcium alkyl benzene sulphonate, triestryl phenol phosphate ester, nonyl phenol ethoxylate, or combination thereof.

4. The composition as claimed in claim 1 wherein said emulsifiers are in an amount of 3-25% w/w.

5. The composition as claimed in claim 1, wherein said solvent bulk medium is carrier oil selected from soya bean oil, sunflower oil, rice bran oil, ground nut oil, castor oil, mineral oils and C9 Aromatic Hydrocarbons Solvents or mixture thereof, and is in an amount of 20-90% w/w.

6. The composition as claimed in claim 1, wherein the composition protects wood from termites, fungi and also acts as termiticide for wood and termite management applications.

7. The composition as claimed in claim 1, wherein the said Emulsifiable concentrate composition is stable at temperatures between 0° C. to 54° C., for a storage period of 12 months.

8. An Emulsifiable Concentrate composition of Cedar wood (Cedrus deodara) oil prepared by a process comprising the steps of:
(i) premixing Cedar wood oil with solvent bulk medium selected from edible oil, non-edible oil, vegetable oil, mineral oil or a combination thereof, at low shear mixing at 40-50 rpm;
(ii) adding at least one emulsifier selected from ionic or non-ionic emulsifiers or a combination thereof to the solution of step (i), with continued low high shear mixing at a speed of 40-50 rpm, to prepare emulsifiable concentrate composition; and
(iii) optionally filtering the emulsified concentrate composition of step (ii) for further purification.

9. The process as claimed in claim 8, wherein said solvent bulk medium in step (i) is carrier oil selected from soya bean oil, sunflower oil, rice bran oil, ground nut oil, castor oil, mineral oils and C9 Aromatic Hydrocarbons Solvents or mixture thereof.

10. The process as claimed in claim 8, wherein said emulsifier of step (ii) is selected from ethoxylated castor oil, tristeryl phenol ethoxylate, fatty alcohol ethoxylate, calcium alkyl benzene sulphonate, triestryl phenol phosphate ester, nonyl phenol ethoxylate, or combination thereof.
